(19) United States
(12) Patent Application Publication
(10) Pub. No.: US 2022/0339059 A1
(43) Pub. Date: Oct. 27, 2022

(54) MASSAGE MODULE WITH IMPROVED DEGREE OF FREEDOM

(71) Applicant: BODYFRIEND CO., LTD., Seoul (KR)

(72) Inventors: Deok Hyun KONG, SEOUL (KR); Do Hyun KIM, Seoul (KR); Kang Min KIM, Seoul (KR)

(21) Appl. No.: 17/730,784

(22) Filed: Apr. 27, 2022

(30) Foreign Application Priority Data
Apr. 27, 2021 (KR) 10-2021-0054579
Apr. 29, 2021 (KR) 10-2021-0055821
Dec. 21, 2021 (KR) 10-2021-0183470
Dec. 21, 2021 (KR) 10-2021-0183471
Dec. 21, 2021 (KR) 10-2021-0183472

(51) Int. Cl. A61H 15/00; A61H 39/04; A61H 23/02; A61H 23/00

(52) U.S. Cl. CPC A61H 15/0078; A61H 39/04; A61H 23/0254; A61H 23/006; A61H 2201/0192; A61H 2201/1671; A61H 2201/0165; A61H 2201/0142; A61H 2203/0456; A61H 2201/164; A61H 2015/005; A61H 15/02

(57) ABSTRACT

Provided is a massage module having an improved degree of freedom, and more particularly, to a massage module having an improved degree of freedom, which, when a force is applied to the massage module, may provide a safe massage to the body of a user by buffering the force. To this end, the massage module may include a massage part that provides a massage to the body of a user, a tilting part that is disposed on one side of the massage part and controls rotation of the massage part, and a height adjustment part that is connected to the tilting part and adjusts a height of the massage part, wherein the massage part includes at least one buffer part disposed at a portion of the massage part that is directly coupled to the tilting part.

### CROSS-REFERENCE TO RELATED APPLICATION

This application claims priority to and the benefit of Korean Patent Application Nos. 10-2021-0054579 filed on Apr. 27, 2021, 10-2021-0055821 filed on Apr. 29, 2021, 10-2021-0183470 filed on Dec. 21, 2021, 10-2021-0183471 filed on Dec. 21, 2021, and 10-2021-0183472 filed on Dec. 21, 2021, the disclosures of which are incorporated herein by reference in their entirety.

### BACKGROUND

### 1. Field of the Invention

The present disclosure relates to a massage module having an improved degree of freedom.

### 2. Discussion of Related Art

A massage is auxiliary medical therapy of applying various types of mechanical stimuli, such as kneading, pressing, pulling, tapping, or moving, to parts of the body of a subject in order to adjust modulation of the body, and is known to help with blood circulation and relieve fatigue of the subject.

An increase in demand for massage has caused an increase in demand for massage devices or massage machines that provide artificial massage functions due to economic reasons and time reasons. That is, as the demand for relaxing knotted muscles through massage in order to relieve fatigue or stress increases, various massage devices that are time-effective and cost-effective have been developed and released.

In recent years, massage devices have been manufactured in a bed type in which a user may receive a massage while lying down. A bed-type massage device may provide a massage by a massage module moving to stimulate the waist and back of the lying user.

Meanwhile, the existing bed-type massage devices have a disadvantage in that the massage devices have a large volume because a length thereof should be at least as long as a height of a user. However, in order to solve this problem, the massage device according to the related art includes a configuration that allows a part corresponding to a leg to be manually folded, but there is an inconvenience in that, whenever the massage is provided, the leg part should be manually operated.

Further, since the massage module that directly provides the massage provides the massage while moving in direct or indirect contact with the body of the user, the massage module may be caught by the body of the user, and in this case, when the massage module is forcibly moved, there is a risk of injuring the body of the user.

Thus, the development of massage devices which may have a reduced volume, safely provide a massage to the body of a user, and further provide a more efficient massage to the body of a user is required.

### SUMMARY OF THE INVENTION

The present disclosure is directed to providing a massage module that, when a force is applied to the massage module, may provide a safe massage to the body of a user by buffering the force.

The present disclosure is also directed to providing a massage module of which the height and angle may be adjusted to correspond to the body of a user.

However, the problems to be solved by the present disclosure are not limited to the above-described problems and include problems that may be easily understood by those skilled in the art to which the present disclosure pertains from the present specification and the accompanying drawings.

According to an aspect of the present disclosure, there is provided a massage module having an improved degree of freedom, including a massage part that provides a massage to the body of a user, a tilting part that is disposed on one side of the massage part and controls rotation of the massage part, and a height adjustment part that is connected to the tilting part and adjusts a height of the massage part, wherein the massage part includes at least one buffer part disposed at a portion of the massage part that is directly coupled to the tilting part.

The tilting part may include a tilting frame connected to the buffer part and an elastic member that controls rotation of the tilting frame.

The elastic member may be a torsion spring that is pressed while rotating about a rotary axis of the tilting frame.

The height adjustment part may include a height adjustment bracket, a first rack gear provided on the height adjustment bracket, a first pinion gear that rotates while engaged with the first rack gear, and a rotation actuator that provides rotational power to the first pinion gear.

The height adjustment bracket may include a guider extending in a lengthwise direction of the first rack gear.

The guider may have a stopper formed at one end thereof.

The buffer part may include a damper member of which a shape is deformed by a force applied to the massage part.

The buffer part may include a damper shaft that is disposed to pass through the damper member and has one end extending inward from the massage part and another end extending to the tilting part.

The massage module may further include a movement part that moves the massage part, the tilting part, and the height adjustment part accommodated in a space that is provided for accommodating the height adjustment part.

### BRIEF DESCRIPTION OF THE DRAWINGS

The above and other objects, features and advantages of the present disclosure will become more apparent to those of ordinary skill in the art by describing exemplary embodiments thereof in detail with reference to the accompanying drawings, in which:

FIG. 1 is a view illustrating a usage example of a massage device according to one embodiment of the present disclosure;

FIG. 2 is a view for describing a driving example of the massage device of FIG. 1;

FIG. 3 is a view for describing driving of a leg support of FIG. 2;

FIG. 4 is a view for describing a driving principle of the leg support of FIG. 3;

FIG. 5 is a view illustrating one embodiment of the massage device including an outer skin member;

FIG. 6 is a view illustrating a massage module according to one embodiment of the present disclosure;

FIG. 7 is a schematic exploded perspective view of FIG. 6;

FIG. 8 is a view illustrating a massage part of FIG. 7;

FIG. 9 is an exploded perspective view illustrating a massage ball of FIG. 8 according to one embodiment;

FIG. 10 is an exploded perspective view illustrating a massage ball of FIG. 8 according to another embodiment;

FIG. 11 is an exploded perspective view illustrating a massage ball of FIG. 8 according to still another embodiment;

FIG. 12 is a view for describing a coupling process of an upper cover of FIG. 8;

FIG. 13 is a view illustrating a buffer part of FIG. 8;

FIG. 14 is a view for describing a function of the buffer part of FIG. 13;

FIG. 15 is a view for describing an embodiment in which a temperature sensor is disposed adjacent to the massage part FIG. 8;

FIG. 16 is a cross-sectional view along line X-X′ of FIG. 15;

FIG. 17 is a view for describing one driving example of a tilting part of FIG. 7;

FIG. 18 is an exploded perspective view of the tilting part of FIG. 7;

FIG. 19 is a view illustrating a height adjustment part of FIG. 7;

FIG. 20 is a view illustrating the height adjustment part of FIG. 19 from a lateral side;

FIG. 21 is a view for describing a driving principle of the height adjustment part of FIG. 19; and

FIG. 22 is a view illustrating a movement part of FIG. 7.

### DETAILED DESCRIPTION OF EXEMPLARY EMBODIMENTS

Objectives, features, and advantages of the present disclosure described above will become more apparent through the following embodiments related to the accompanying drawings. The following specific structure or functional descriptions are exemplified only to describe embodiments according to the concept of the present disclosure, the embodiments according to the concept of the present disclosure may be implemented in various forms, and the present disclosure should not be construed as being limited to the embodiments described in the present specification or application.

Since the embodiments according to the concept of the present disclosure may be variously changed and have various forms, specific embodiments are intended to be illustrated in the drawings and to be described in detail in the present specification or application. However, it should be understood that this is not intended to limit the embodiments according to the concept of the present disclosure to a specific disclosure, and all changes, equivalents, and substitutes included in the spirit and scope of the present disclosure are included.

Terms such as “first” and/or “second” may be used to describe various components, but the components are not limited by the terms. The terms are used only to distinguish one component from other components. For example, without departing from the scope of rights according to the concept of the present disclosure, a first component may be called a second component, and similarly, a second component may be called first component.

When it is described that one element is “connected to” or “electrically connected to” another element, it should be understood that the first element may be directly connected or electrically connected to the second element or a third element may be provided therebetween. On the other hand, when it is described that an element is “directly connected to” or “directly electrically connected to” another element, it should be understood that no third element is present therebetween. Other expressions used to describe the relationships between components, such as “between” and “directly between” or “adjacent” and “directly adjacent,” should be interpreted in the same manner.

Terms used in the present specification are used only to describe specific embodiments and are not intended to limit the present disclosure. Singular expressions include plural expressions unless clearly otherwise indicated in the context. It should be understood in the present specification that the terms “include” or “have” are intended to indicate that there are features, numbers, steps, operations, components, parts, or combinations thereof that are implemented and do not exclude in advance the possibility of the presence or addition of one or more other features, numbers, steps, operations, components, parts, or combinations thereof.

Unless otherwise defined, all terms used herein including technical or scientific terms have the same meanings as those commonly understood by those skilled in the art to which the present disclosure belongs. Terms defined in commonly used dictionaries should be interpreted as having the same meanings in the context of the related art and should not be interpreted with ideal or excessively formal meanings, unless explicitly defined in the present specification.

In the present specification, an actuator is a component that may provide a driving force. For example, examples of actuators may include a motor, a linear motor, an electronic motor, a direct current (DC) motor, an alternating current (AC) motor, a linear actuator, an electric actuator, and the like, but the present disclosure is not limited thereto.

Hereinafter, embodiments of the present disclosure will be described in detail with reference to the accompanying drawings.

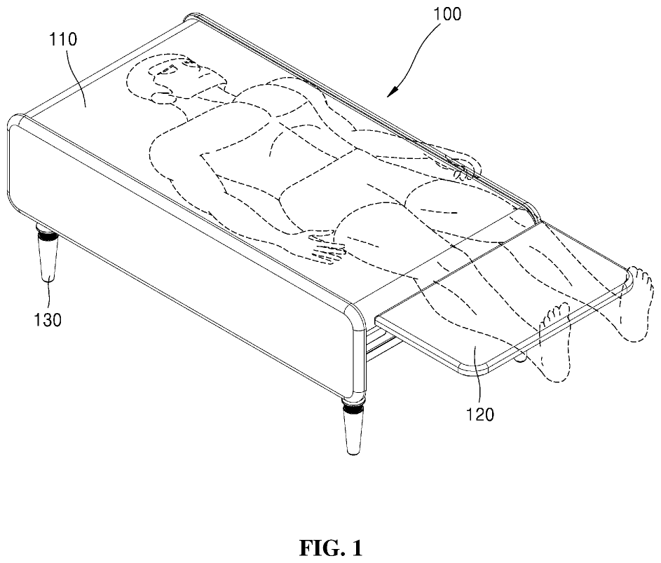

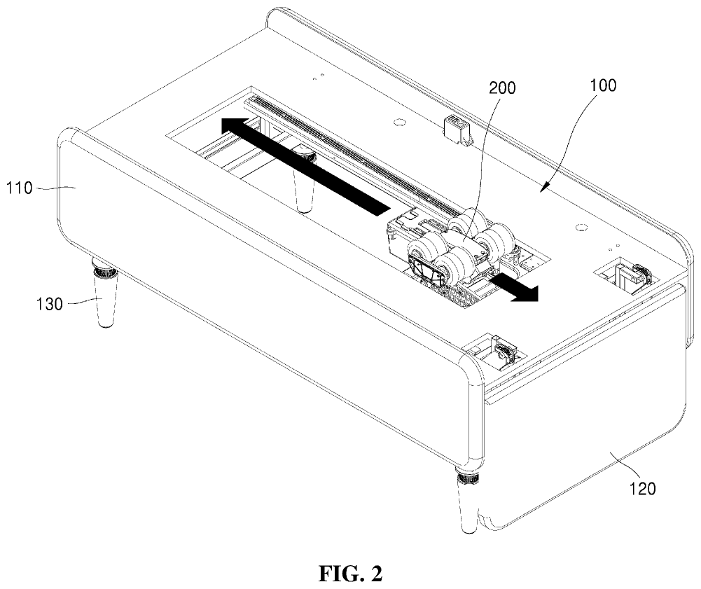

FIG. 1 is a view illustrating a usage example of a massage device 100 according to one embodiment of the present disclosure, and FIG. 2 is a view for describing a driving example of the massage device 100 of FIG. 1.

Referring to FIGS. 1 and 2, the massage device 100 according to the present disclosure may be a bed-type massage device.

The massage device 100 may include a body part 110 and a massage module 200.

The body part 110 may form the entire outer shape of the massage device 100. The massage module 200 may be disposed in the massage device 100 to provide a massage to the body of a user.

The entirety of body part 110 may be formed in a substantially box shape, and for example, may be formed in the shape of a bed.

The body part 110 may include an upper surface on which the user may lie. For example, the user may receive a massage while lying with his/her back on the upper surface of the body part 110.

A space may be provided inside the body part 110. For example, the body part 110 may have the shape of a bed when viewed from the outside, and the space may be provided thereinside. To this end, a frame constituting the entire shape of the body part 110 may be disposed inside the body part 110, and at least one plate may be disposed on the basis of the frame to configure the body part 110.

The massage module 200 may be disposed inside the body part 110. For example, the massage module 200 may be disposed in the space provided inside the body part 110.

The massage module 200 may provide the massage to the user while in direct or indirect contact with the body of the user.

The massage module 200 may provide a massage by applying acupressure to the body of the user or applying vibration to the body of the user.

For example, the massage module 200 may provide an acupressure massage by applying the acupressure to the body of the user while moving in contact with the body of the user.

Alternatively, the massage module 200 may include a vibration motor, which will be described below, to provide a vibration massage to the body of the user.

Alternatively, the massage module 200 may provide a tapping massage while vertically moving at a predetermined location.

However, the modes of the massage provided by the massage module 200 are not limited thereto.

The massage module 200 will be described in detail below.

A massage groove 111 may be formed in the upper surface of the body part 110 so that at least a portion of the massage module 200 is exposed.

As one embodiment, the massage groove 111 may be formed to have a length in a direction of a height of the user.

Referring back to FIG. 2, the massage module 200 may be disposed inside the body part 110, and the at least a portion of the massage module 200 may be exposed to the outside through the massage groove 111 formed in the upper surface of the body part 110.

The at least a portion of the massage module 200 may be exposed to the outside through the massage groove 111, and thus the massage module 200 may provide a massage while in direct or indirect contact with the body of the user.

Here, a state in which the massage module 200 provides a massage while in direct contact with the body of the user may mean that the massage device 100 does not include a separate member covering the massage groove 111, and thus the massage module 200 provides a massage while in direct contact with the body of the user.

Further, a state in which the massage module 200 provides a massage while in indirect contact with the body of the user means that the massage device 100 includes a separate member for covering the massage groove 111. In detail, this state means that the separate member for covering the massage groove 111 is disposed between the massage module 200 and the body of the user. Thus, the massage module 200 may apply the acupressure to the separate member for covering the massage groove 111 or apply vibration to the member, and the acupressure or the vibration may be transferred to the body of the user. In this case, the separate member for covering the massage groove 111 may be fabric, leather, a synthetic resin, a synthetic leather, or the like, but the present disclosure is not limited thereto.

The massage module 200 may move in a longitudinal direction of the massage groove 111 inside the body part 110. For example, the massage module 200 may provide a massage to the body of the user while moving along the massage groove 111.

Thus, the massage module 200 may provide a massage to various portions of the body of the user. In detail, the massage module 200 may provide a massage to the body of the user while moving from a portion of the user adjacent to the neck to the waist of the user.

As one embodiment, the massage device 100 may include a leg support 120 that is disposed on one side of the body part 110 and supports at least a portion of a leg of the user.

The leg support 120 may support a leg portion that is not supported by the body part 110 in a state in which the user lies on the upper surface of the body part 110.

The massage device 100 according to the present disclosure may further include a controller. The controller may control an operation of the massage device 100. The controller may be implemented as one processor or may be implemented as a plurality of processors. When the controller is implemented as the plurality of processors, at least some of the plurality of processors may be located physically apart from each other. Further, the controller is not limited thereto and may be implemented in various manners.

For example, the controller may correspond to one or a plurality of processors. The processor may be implemented as an array of a plurality of logic gates or may be implemented as a combination of a general-purpose microprocessor and a memory in which a program executable in the microprocessor is stored. Further, it may be understood by those skilled in the art to which the present embodiment pertains that the controller may be implemented in other types of hardware.

According to one embodiment, the controller may control operation of the massage device 100. For example, the controller may adjust a rotational angle of the leg support 120, which will be described below. Further, the controller may control an operation of the massage module 200. In detail, the controller may control the massage module 200 to move in the direction of the height of the user and control a height of the massage module 200 to be adjusted. Further, as will be described below, the controller may control the intensity, a heating temperature, and the like of the massage provided to the user by the massage module 200.

Further, the massage device 100 may include a plurality of actuators, and the massage device 100 may control the operation of the massage device 100 by controlling the plurality of actuators.

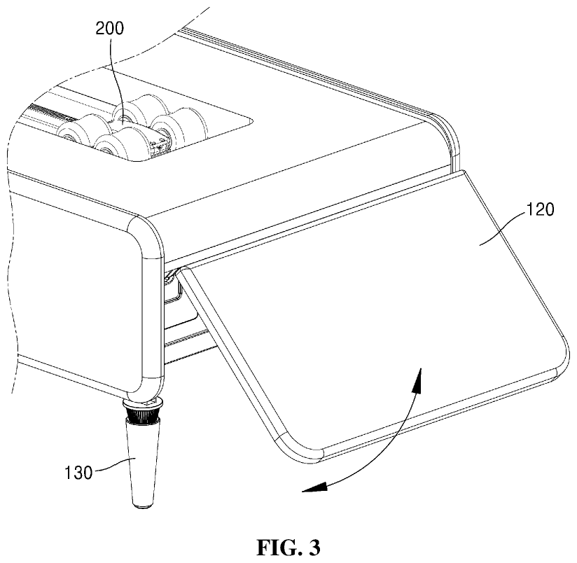

FIG. 3 is a view for describing driving of the leg support 120 of FIG. 2.

Referring to FIG. 3, as one embodiment, the leg support may rotate at least in one direction. For example, the leg support 120 may be unfolded in a direction parallel to the upper surface of the body part 110 or folded in a direction toward the ground according to an operation of the user.

When the leg support 120 is unfolded in the direction parallel to the upper surface of the body part 110, the leg support 120 may support the leg of the user while the user lies on the upper surface of the body part 110.

When the leg support 120 is folded in the direction toward the ground, the leg support 120 is not unfolded outward from one side of the body part 110, and thus the entire volume of the massage device 100 may be reduced. Thus, when the user does not use the massage device 100, the leg support 120 may be stored in a folded state.

Alternatively, the user may rest while sitting or lying on the body part 110 in a state in which the leg support 120 is folded and may receive a massage in a state in which the leg support 120 is folded according to selection.

As one embodiment, a location of the leg support 120 may be freely determined within a predetermined angle range according to the operation of the user. The user may use the massage device 100 in a state in which the leg support 120 is fixed to a specific location between the direction parallel to the upper surface of the body part 110 and the direction toward the ground.

In detail, the leg support 120 may freely rotate in the range of 0° to 90° with respect to an angle formed between the leg support 120 and the upper surface of the body part 110 and may be fixed to a specific location in the range of 0° to 90°. Thus, the user may use the massage device 100 after rotating the leg support 120 according to a shape of the body, characteristics, or tendencies of the user himself/herself.

In this case, in order to freely rotate and fix the leg support 120 in the range of 0° to 90°, it is preferable not to manually move the leg support 120 but to control the rotation of the leg support 120 by a power transmission unit.

For example, in order to manually adjust the angle of the leg support 120, a method in which, after the leg support 120 is lifted up, a boss and the like is hung on a specific stopping step should be used. However, in this case, a location in which the leg support 120 may be fixed is limited according to the location and the number of the stopping steps.

To this end, the massage device 100 according to one embodiment of the present disclosure may further include a unit for controlling the rotation of the leg support 120.

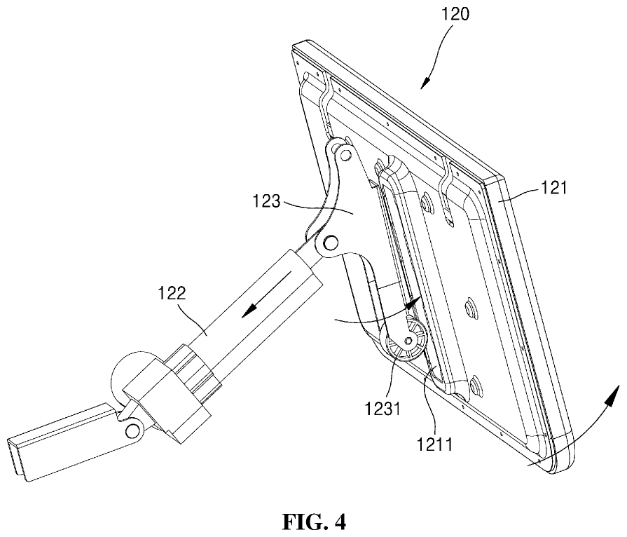

FIG. 4 is a view for describing a driving principle of the leg support 120 of FIG. 3.

Referring to FIG. 4, the leg support 120 may include a leg support plate 121 and a leg actuator 122.

The leg support plate 121 may be configured to support the leg of the user while in contact with the leg of the user. For example, the leg support plate 121 may be substantially formed in the shape of a plate. The user may receive a massage in a state in which the at least a portion of the leg of the user himself/herself is placed on the leg support plate 121.

The leg actuator 122 may be a part that provides power for rotating the leg support plate 121 to the leg support plate 121.

As one embodiment, the leg actuator 122 may be a linear actuator. Thus, the leg actuator 122 may rotate the leg support plate 121 while linearly extending and contracting.

In this case, the leg actuator 122 may be controlled by the controller. Further, the user may input a signal for controlling the leg actuator 122 to the controller through a separately provided input unit.

As one embodiment, the leg support 120 may further include a link member 123 that transfers power generated from the leg actuator 122 to the leg support plate 121. The link member 123 may be a part for rotating the leg support plate 121 by performing a rotational movement by a linear movement of the leg actuator 122.

As a selective embodiment, a guide rail 1211 may be formed on one surface of the leg support plate 121 to have a length, and a wheel member 1231 that moves along the guide rail 1211 may be disposed on one side of the link member 123.

As a specific embodiment, a front surface of the leg support plate 121 may support the leg of the user while in contact with the leg of the user. Further, the guide rail 1211 may be formed on a rear surface of the leg support plate 121. The guide rail 1211 may be formed on the rear surface of the leg support plate 121 in the longitudinal direction.

The wheel member 1231 may be disposed on one side of the link member 123, and a separately provided rotary shaft may be connected to the other end of the link member 123. In this case, the link member 123 may rotate about the connected rotary shaft. Further, a portion of the link member 123, for example, a specific portion between one end and the other end of the link member 123, may be connected to the leg actuator 122.

Thus, when the operation of the leg support 120 is described with reference to FIG. 4, and when the leg actuator 122 is linearly extended, the link member 123 may be pushed in a direction opposite to the linear actuator 122, and accordingly, the link member 123 may rotate in a counterclockwise direction about the connected rotary shaft. In this case, the wheel member 1231 may move along the guide rail 1211 while rotating and may move from a lower portion toward an upper portion of the guide rail 1211. As the wheel member 1231 moves along the guide rail 1211, the leg support plate 121 may be lifted up while rotating in the counterclockwise direction due to the rotation of the link member 123 and the movement of the wheel member 1231.

In contrast, when the leg actuator 122 is linearly contracted with reference to FIG. 4, the link member 123 may be pulled in a direction toward the linear actuator 122, and accordingly, the link member 123 may rotate in a clockwise direction about the connected rotary shaft. In this case, the wheel member 1231 may move along the guide rail 1211 while rotating and may move from the upper portion toward the lower portion of the guide rail 1211. As the wheel member 1231 moves along the guide rail 1211, the leg support plate 121 may be folded while rotating in the clockwise direction due to the rotation of the link member 123 and the movement of the wheel member 1231.

As a selective embodiment, the massage device 100 may further include a leg part 130 disposed below the body part 110. The leg part 130 is a part disposed below the body part 110 to support the massage device 100.

The leg part 130 may be formed to have a length. Thus, the massage device 100 may be spaced a predetermined distance from the ground by the leg part 130. Since the massage device 100 is spaced apart from the ground, a predetermined space may be provided, and the user may utilize this space as a storage space or the like.

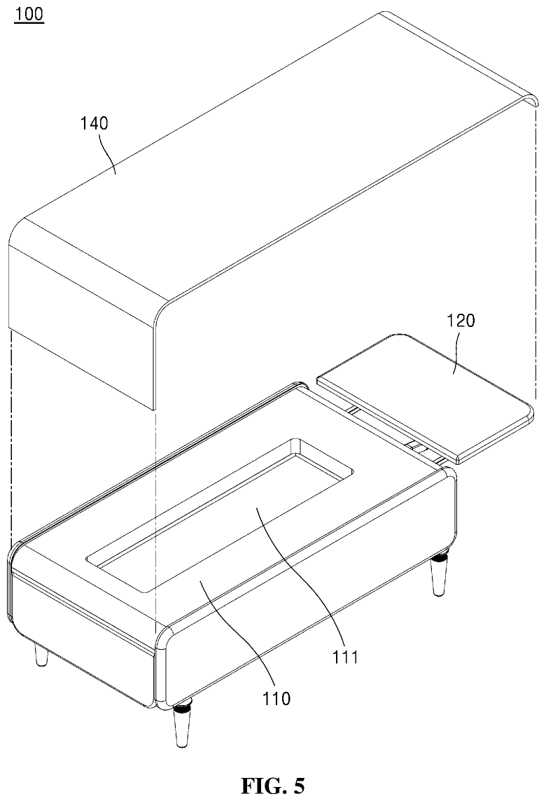

FIG. 5 is a view illustrating one embodiment of the massage device 100 including an outer skin member 140.

Referring to FIG. 5, the massage device 100 according to one embodiment of the present disclosure may further include the outer skin member 140.

The outer skin member 140 may be a part that covers at least a portion of the massage device 100. For example, the outer skin member 140 may cover at least a portion of the body part 110. As another example, the outer skin member 140 may cover the entire body part 110.

Since the massage device 100 is a device that is used while the user lies thereon and is used for a long time without replacement, there is a risk of contamination or damage.

Thus, the outer skin member 140 may protect the massage device 100 from the contamination by covering the at least a portion of the body part 110. Further, the outer skin member 140 may reduce the risk of damage by covering the at least a portion of the body part 110.

Further, as will be described below, one embodiment of the massage module 200 used in the present disclosure may provide heat to the body of the user. In this case, sweat may occur in the body of the user, and the massage device 100 may be contaminated by the sweat.

To this end, as a selective embodiment, the outer skin member 140 may be detachably attached to the body part 110. Thus, after using the massage device 100, the user may separate the outer skin member 140 from the body part 110 and then wash the outer skin member 140.

As illustrated in FIG. 5, when the outer skin member 140 is disposed to cover all the upper surface of the body part 110 and the leg support 120, the outer skin member 140 should fold and unfold with rotation of the leg support 120.

Thus, the outer skin member 140 may be formed of a material that is easily deformable. For example, the outer skin member 140 may be made of fabric, leather, synthetic leather, or the like, but the present disclosure is not limited thereto.

As a selective embodiment, the outer skin member 140 may have a length that is greater than a value obtained by adding the length of the upper surface of the body part 110 and the length of the leg support 120.

For example, the lengths of the outer skin member 140 that are required when the leg support 120 is unfolded and when the leg support 120 is folded toward the ground are different from each other. That is, the required length of the outer skin member 140 is greater when the leg support 120 is folded than when the leg support 120 is unfolded. Thus, the outer skin member 140 may be formed to be longer than a value obtained by adding the length of the upper surface of the body part 110 and the length of the leg support 120 so that the length is not insufficient, and the outer skin member 140 may completely cover the leg support 120 and the body part 110 even when the leg support 120 is folded.

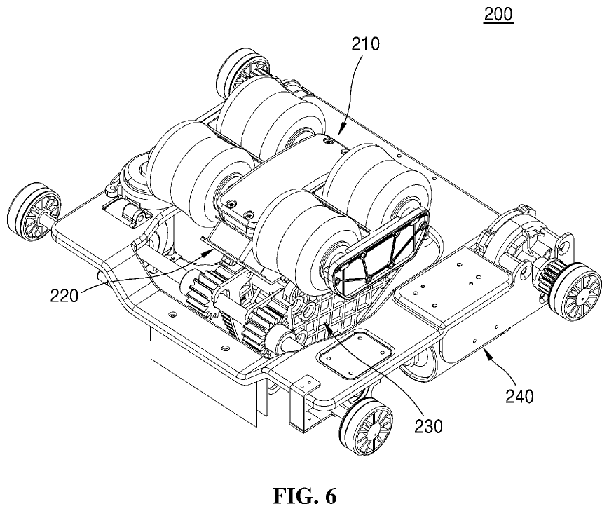

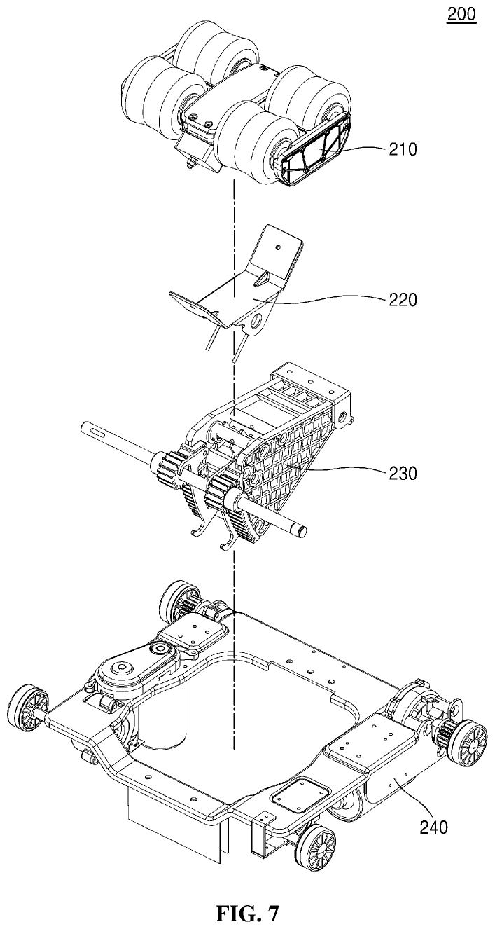

FIG. 6 is a view illustrating a massage module 200 according to one embodiment of the present disclosure, and FIG. 7 is a schematic exploded perspective view of FIG. 6.

Referring to FIGS. 6 and 7, the massage module 200 according to one embodiment of the present disclosure may include at least one of a massage part 210, a tilting part 220, a height adjustment part 230, and a movement part 240.

The massage part 210 may be a part that provides a massage to the body of the user.

For example, the massage part 210 may provide a vibration massage, an acupressure massage, a tapping massage, and the like to the body of the user.

A configuration of the massage part 210 will be described below in detail.

The tilting part 220 is a part that is disposed on one side of the massage part 210 and controls rotation of the massage part 210.

For example, the tilting part 220 may be coupled to the massage part 210 and control the rotation of the massage part 210. When an external force is applied to the massage part 210, the tilting part 220 may prevent damage by rotating the massage part 210 to an appropriate angle and allow the massage part 210 to safely provide a massage to the body of the user.

A configuration of the tilting part 220 will be described below in detail.

The height adjustment part 230 may be a part that is connected to the tilting part 220 and adjusts the height of the massage part 210.

For example, the height adjustment part 230 may be connected to the tilting part 220, and the tilting part 220 may be connected to the massage part 210. Thus, the height adjustment part 230 may adjust the height of the tilting part 220, and at the same time, adjust the height of the massage part 210.

Accordingly, the height of the massage part 210 may be adjusted to correspond to the body of the user, and the massage part may provide a massage to the body of the user.

Further, the height of the massage part 210 may be adjusted to correspond to an intensity of a massage to be provided, and thus the massage part 210 may provide massages having various intensities to the body of the user.

A configuration of the height adjustment part 230 will be described below in detail.

The movement part 240 may be a part that moves the massage module 200. For example, the movement part 240 may be provided with a space for accommodating the height adjustment part 230, and the height adjustment part 230 may be accommodated in the provided space. In this case, the height adjustment part 230 may be connected to the tilting part 220, and the tilting part 220 may be connected to the massage part 210. Thus, the movement part 240 may move the accommodated height adjustment part 230 to move the massage part 210, the tilting part 220, and the height adjustment part 230. That is, the movement part 240 may move the entire massage module 200.

A configuration of the movement part 240 will be described below in detail.

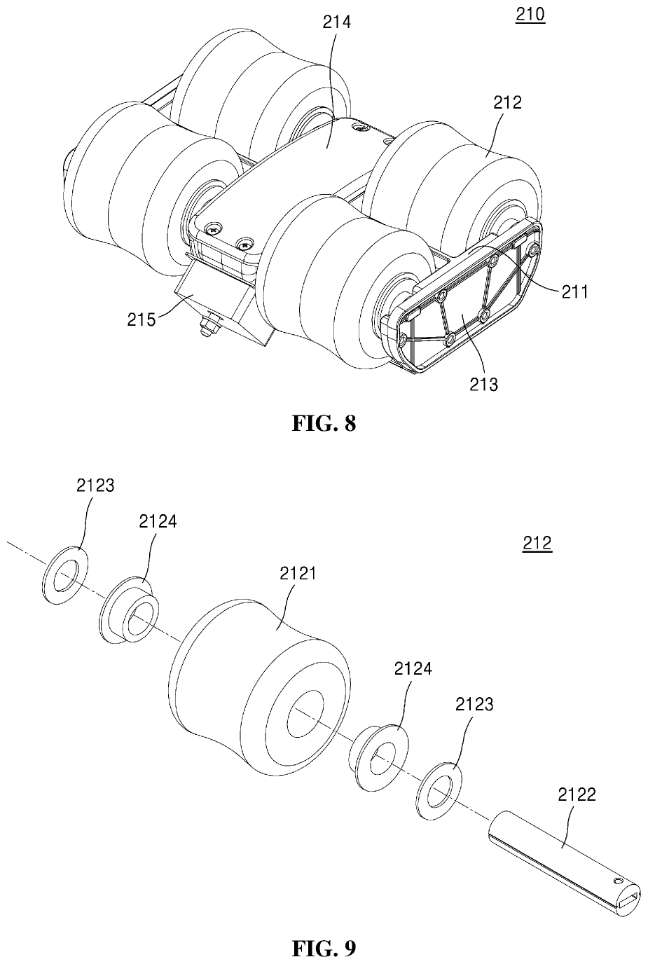

FIG. 8 is a view illustrating the massage part 210 of FIG. 7.

Referring to FIG. 8, the massage part 210 may include at least one of a module base 211, a massage ball 212, a side cover 213, and an upper cover 214.

The module base 211 may form the entire frame of the massage part 210. The module base 211 may be provided with a space for accommodating the massage ball 212. As will be described below, the massage part 210 may include at least one massage ball 212, and the module base 211 may be provided with spaces for accommodating the massage balls 212 to correspond to the number of provided massage balls 212.

The side covers 213 may be arranged on both sides of the module base 211. In detail, as will be described below, the massage ball 212 may roll along the body of the user, and a member serving as an axis for a rotational movement of the massage ball 212 may be fixed by the side cover 213. Thus, a rotary shaft of the massage ball 212 may be prevented from being separated from the massage part 210.

Although not illustrated, the massage part 210 may further include a vibration motor. The vibration motor may generate vibrations. The vibration motor may transfer the generated vibrations to the massage ball 212. The massage ball 212 may provide a vibration massage by transferring the vibrations to the body of the user.

As one embodiment, the massage ball 212 may further include a heating member 2122.

The heating member 2122 may be a part that generates heat by supplied power. In this case, the generated heat may be transferred to the massage ball 212. The massage ball 212 may provide a heating massage to the user through the transferred heat while in contact with the body of the user.

FIG. 9 is an exploded perspective view illustrating the massage ball 212 of FIG. 8 according to one embodiment.

Referring to FIG. 9, the massage ball 212 may include a ball member 2121 and the heating member 2122.

The massage ball 212 may be a member that is in direct or indirect contact with the body of the user. For example, the massage ball 212 may be a part that provides a massage to the user while in contact with the body of the user.

The heating member 2122 may generate heat by supplied power. The generated heat may be transferred to the ball member 2121.

The ball member 2121 may be formed of a thermally conductive material. Thus, the heat generated from the heating member 2122 may be transferred to the ball member 2121, and the ball member 2121 may provide a heating massage by transferring the transferred heat to the body of the user.

As an example, the ball member 2121 may be formed of a carbon material. As a specific example, the ball member 2121 may be formed of a carbon fiber. Thus, the ball member 2121 may radiate far infrared rays, may easily receive the heat generated by the heating member 2122, and may easily transfer the received heat to the body of the user.

As one embodiment, at least a portion of the heating member 2122 may be inserted into the ball member 2121. The heating member 2122 may generate the heat while inserted into the ball member 2121 and transfer the heat in all directions inside of the ball member 2121. Thus, the ball member 2121 may efficiently receive the heat from the heating member 2122.

As a selective embodiment, the heating member 2122 may be formed in a substantially cylindrical shape. The heating member 2122 may be inserted through the ball member 2121 and may serve as a rotary shaft about which the ball member 2121 rotates.

That is, the ball member 2121 may rotate about the heating member 2122 when providing a massage to the body of the user. In this case, the heating member 2122 may continuously transfer the heat to the ball member 2121. Thus, heat transfer efficiency may be further improved.

The heating member 2122 may include a housing and a heat source.

The housing may cover the heat source. For example, the housing may have a space provided therein and may have a substantially cylindrical shape. The heat source may be disposed in the space provided inside the housing.

The heat source may be a part that generates heat by supplied power. For example, the heat source may have an electrical resistance. Thus, when power is supplied from the outside and electricity flows, the heat source may generate heat.

As an example, the heat source may be a nichrome wire. As another example, the heat source may be a positive temperature coefficient (PTC) heater. However, the present disclosure is not limited thereto.

The heating member 2122 may be manufactured by crushing at least a portion of the housing after the heat source is disposed inside the housing. In this case, a portion of the heating member 2122 may fix the heat source while being crushed, and the remaining portion may be maintained in a substantially cylindrical shape.

When the heating member 2122 is manufactured in this way, the heat source may be firmly fixed to the housing, and the heating member 2122 may be easily manufactured.

The amount of heat generated by the heating member 2122 may be controlled by the controller. For example, the user may input the amount of heat generated by the heating member 2122 or a desired temperature to the controller through a separately provided input unit. By this input, the controller may control the power supplied to the heating member 2122 to control the amount of heat generated by the heating member 2122.

As a selective embodiment, the massage ball 212 may further include a washer 2124 disposed on a side surface of the ball member 2121. As a preferable embodiment, the washers 2124 may be arranged on both sides of the ball member 2121. Accordingly, the ball member 2121 may stably rotate about the heating member 2122. Further, the washer 2124 may protect a side surface of the ball member 2121.

As an example, the washer 2124 may be made of polyoxymethylene (POM). Thus, the washer 2124 may have a physical property that makes it strong against friction or impact and allow it to protect the ball member 2121.

As a selective embodiment, the massage ball 212 may further include a bearing 2123 disposed at a location adjacent to the washer 2124. In this case, the bearing 2123 may be formed of a thermally conductive material. Thus, the heat generated by the heating member 2122 may also be transferred to the bearing 2123, and the bearing 2123 may transfer the transferred heat to the ball member 2121.

Accordingly, the heat generated by the heating member 2122 may be directly transferred to the ball member 2121 at a portion thereof in contact with the ball member 2121 and may be transferred to the ball member 2121 through the bearing 2123 at a portion thereof that protrudes outward from the ball member 2121. Thus, heat transfer efficiency may be further improved.

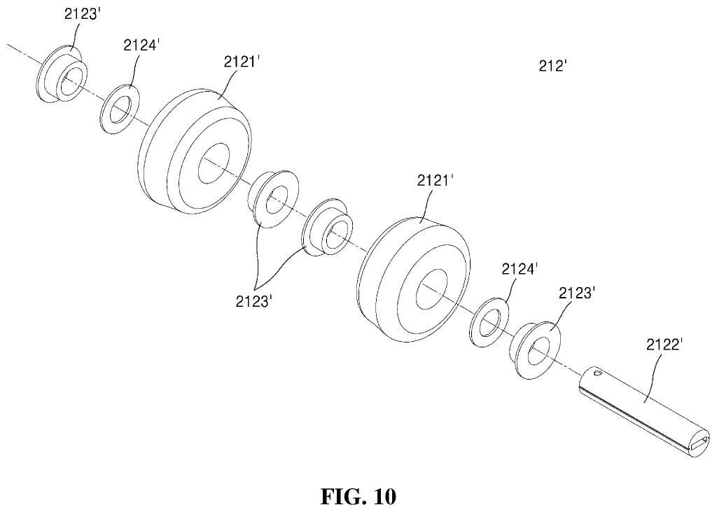

FIG. 10 is an exploded perspective view illustrating a massage ball 212′ of FIG. 8 according to another embodiment.

For convenience of description, a difference between the massage ball 212 described with reference to FIG. 9 and the massage ball 212′ will be mainly described. For example, unless otherwise described below, the configuration described with reference to FIG. 9 may be used without change even for the massage ball 212′ of FIG. 10 or employed within a range that may be easily modified and applied by those skilled in the art.

Referring to FIG. 10, the massage ball 212′ according to the present embodiment may include a ball member 2121′ and a heating member 2122′.

As a selective embodiment, the massage ball 212′ may include washers 2124′ arranged on both sides of the ball member 2121′.

As a selective embodiment, the massage ball 212′ may further include a bearing 2123′ disposed adjacent to the washer 2124′.

As one embodiment, the ball member 2121′ may be formed by coupling two members.

In this way, when the ball member 2121′ is formed by coupling the two members, the respective members constituting the ball member 2121′ may individually rotate. Thus, when the ball member 2121′ rotates in contact with the body of the user, the two members may provide a massage to correspond to the body of the user while rotating. For example, a member in strong contact with the body of the user may provide a massage while strongly and rapidly rotating, and a member in relatively weak contact with the body of the user may provide a massage while weakly and slowly rotating.

As a selective embodiment, at least one washer 2124′ may be disposed between the two members. Thus, when the two members individually rotate, the respective members may be protected from friction or impact by the washer 2124′.

Although not illustrated, as a selective embodiment, the bearing 2123′ may be further disposed between the two members. Thus, heat generated by the heating member 2122′ may also be transferred to the bearing 2123′, the heat transferred to the bearing 2123′ may be transferred to the respective members constituting the ball member 2121′, and thus the heat may be more efficiently transferred.

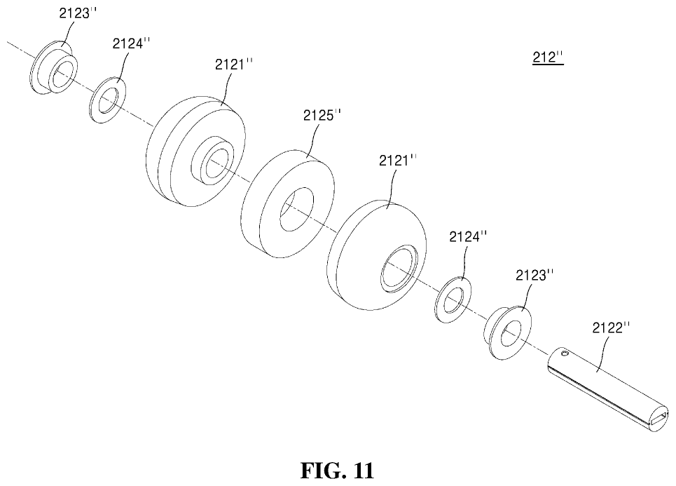

FIG. 11 is an exploded perspective view illustrating a massage ball 212″ of FIG. 8 according to still another embodiment.

For convenience of description, a difference between the massage balls 212 and 212′ described with reference to FIGS. 9 and 10 and the massage ball 212′ will be mainly described. For example, unless otherwise described below, the configurations described with reference to FIGS. 9 and 10 may be used without change even for the massage ball 212″ of FIG. 11 or employed within a range that may be easily modified and applied by those skilled in the art.

Referring to FIG. 11, the massage ball 212″ according to the present embodiment may include a ball member 2121″ and a heating member 2122″.

As a selective embodiment, the massage ball 212″ may include washers 2124″ arranged on both sides of the ball member 2121″.

As a selective embodiment, the massage ball 212″ may further include a bearing 2123″ disposed adjacent to the washer 2124″.

As one embodiment, the ball member 2121″ may be formed by coupling two members.

As a selective embodiment, the massage ball 212″ according to the present embodiment may further include a magnet member 2125″ disposed between the two members.

The magnet member 2125″ may be a permanent magnet and provide a magnetic field therapy effect to the body of the user.

For example, the magnet member 2125″ may interlock with a magnetic field generation module, which will be described below, to provide the magnetic field therapy effect to the user.

Although not illustrated, the massage part 210″ may further include at least one magnetic field generation module. For example, the magnetic field generation module may be located adjacent to the massage ball 212″.

The magnetic field generation module may generate a pulsed electro-magnetic field (PEMF). The PEMF may act on the body of the user to help with regeneration of cells and tissues by inducing intracellular activation. Further, the PEMF may act on the body of the user to activate tissue metabolism of the body, relax muscle tension, and help the spine relax.

Thus, the magnetic field generation module may generate the PEMF to additionally provide the magnetic field therapy effect to the body of the user.

Further, the magnetic field generation module may interlock with the magnet member 2125″ provided in the massage ball 212″ to more efficiently provide the magnetic field therapy to the body of the user.

Further, the magnetic field generation module may generate heat. For example, the magnetic field generation module may generate radiant heat while performing magnetic field therapy. In this case, since the magnetic field generation module is disposed at a location adjacent to the massage ball 212″ as described above, the heat generated by the magnetic field generation module may be transferred to the massage ball 212″. Thus, the magnetic field generation module may function as an additional heat source. That is, since the heat is additionally generated by the magnetic field generation module, power consumption of the heating member 2122″ may be reduced.

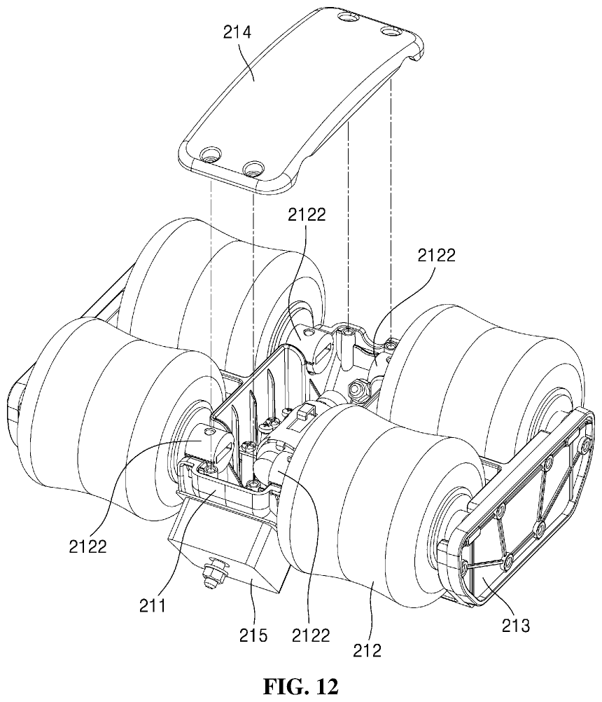

FIG. 12 is a view for describing a coupling process of an upper cover 214 of FIG. 8.

Referring to FIG. 12, the upper cover 214 may be disposed above the module base 211. For example, the massage balls 212 may be arranged on both sides of the module base 211, and the upper cover 214 may be coupled to an upper portion of a space formed between the massage balls 212.

The upper cover 214 may fix the heating member 2122. For example, the heating member 2122 may serve as a rotary shaft of each of the massage balls 212, and the heating member 2122 may be fixed by the upper cover 214 so that a location thereof is not changed.

As a selective embodiment, a groove may be formed at an end of the heating member 2122, and a boss inserted into the groove formed in the heating member 2122 may be formed in the upper cover 214. Thus, as the upper cover 24 is coupled to an upper portion of the module base 211, the boss is inserted into the groove of the heating member 2122, and thus the heating member 2122 may be fixed.

As a selective embodiment, when coupled to the upper portion of the module base 211 to fix the heating member 2122, the upper cover 214 may be disposed at a place lower than the height of the massage ball 212. For example, when the massage part 210 comes into contact with the body of the user, the massage ball 212 comes into contact with the body of the user, and thus the upper cover 214 may be spaced apart from the body of the user. Since the massage ball 212 directly provides a massage such as acupressure, vibration, or tapping to the body of the user, the upper cover 214 is disposed at a location lower than the massage ball 212, and thus may not hinder the massage ball 2112 from providing a massage to the body of the user.

As a selective embodiment, the upper cover 214 may perform an additional heating function. For example, the upper cover 214 may be formed of a thermally conductive material.

Since the upper cover 214 may be connected to the heating member 2122 to fix the heating member 2122, the heat generated by the heating member 2122 may also be transferred to the upper cover 214. In this case, when the massage part 210 provides a heating massage to the body of the user, a certain amount of radiant heat may be transferred from the upper cover 214 to the body of the user.

Accordingly, the heat may be transferred to a portion of the body of the user, which is not in contact with the massage ball 212, by the upper cover 214. Thus, the generated thermal energy may be used as efficiently as possible.

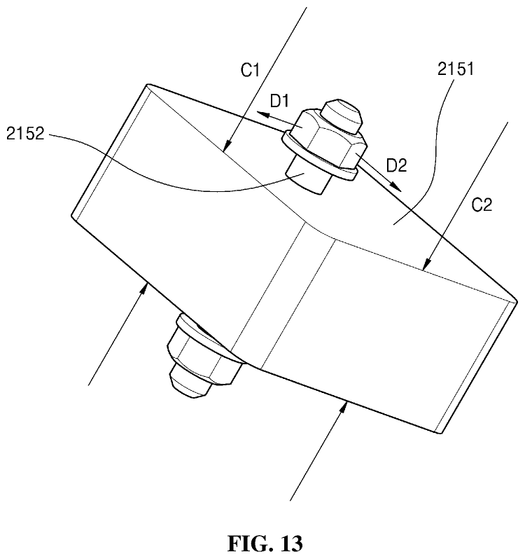

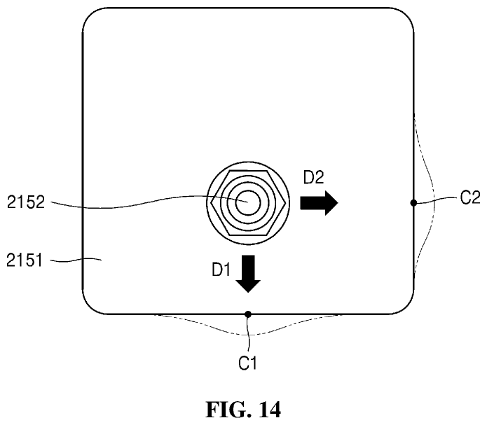

FIG. 13 is a view illustrating a buffer part 215 of FIG. 8, and FIG. 14 is a view for describing a function of the buffer part 215 of FIG. 13.

Referring to FIGS. 13 and 14, the massage part 210 may include at least one buffer part 215.

For example, the buffer part 215 may be disposed below the massage part 210 as shown in FIGS. 7 and 12, and in detail, the buffer part 215 may be disposed at a portion of the massage part 210 that is directly coupled to the tilting part 220. That is, the buffer part 215 may be disposed between the module base 211 and the tilting part 220.

The buffer part 215 may include a damper member 2151 and a damper shaft 2152.

The damper member 2151 may be a part that improves a degree of freedom (DOF) of the massage part 210. The damper member 2151 may be a part that damps a force applied to the massage part 210.

As one embodiment, the damper member 2151 may be formed of an elastic material. For example, the shape of the damper member 2151 may be changed to correspond to an external force when the external force is applied and may return to its original shape when the external force is removed.

As one embodiment, the damper member 2151 may be made of rubber. As a preferable embodiment, the rubber forming the damper member 2151 may have a hardness of 45 to 48 degrees. Accordingly, the rubber forming the damper member 2151 may have sufficient hardness to maintain the entire shape. Further, the rubber forming the damper member 2151 may have sufficient hardness to be deformed by the force applied to the massage part 210.

In this way, the damper member 2151 of the buffer part 215 may maintain the entire shape and may be deformed by the force applied from the outside, thereby improving the degree of freedom of the massage part 210.

The damper shaft 2152 may be a shaft disposed to pass through the damper member 2151.

As one embodiment, the damper shaft 2152 may be a linear coupling member. For example, the damper shaft 2152 may be a screw, a nail, or a bolt, but the present disclosure is not limited thereto.

The damper shaft 2152 may be disposed to pass through the damper member 2151, and both ends thereof may be formed to protrude to the outside of the damper member 2151. One end of the damper shaft 2152 may be coupled to the module base 211. The other end of the damper shaft 2152 may be coupled to the tilting part 220.

That is, the damper member 2151 may be disposed between the module base 211 of the massage part 210 and the tilting part 220, and the module base 211, the damper member 2151, and the tilting part 220 may be connected to each other by the damper shaft 2152.

When a force is applied to the massage part 210, the force may be transferred to the damper shaft 2152 directly connected to the module base 211 and may be transferred to the damper member 2151 through the damper shaft 2152. That is, the force applied to the massage part 21 is to move the damper shaft 2152, the damper shaft 2152 changes an angle while the damper member 2151 is deformed by the force, and thus the degree of freedom of the massage part 210 may be improved.

Referring back to FIGS. 13 and 14, for example, when the damper shaft 2152 is to move in a direction D1 by the force applied to the massage part 210 from the outside, the damper member 2151 may be schematically contracted in a direction C1. When the damper shaft 2152 is to move in a direction D2 by the force applied to the massage part 210 from the outside, the damper member 2151 may be substantially contracted in a direction C2.

Accordingly, a specific portion of the buffer part 215 is contracted by the force applied to the massage part 210, and thus the degree of freedom of the massage part 210 may be greatly improved.

Thus, the massage part 210 may implement the degree of freedom in directions of three axes, that is, 6 DOF, by the buffer part 215. In detail, the massage part 210 may implement movement in a space rather than movement on a plane.

Further, when the massage module 200 provides a vibration massage to the buffer part 215, vibrations generated by a vibration motor may be offset by the buffer part 215. For example, since the buffer part 215 is disposed below the massage part 210, the vibrations generated by the vibration motor may be directly transferred to the massage ball 212 but may be offset while passing through the buffer part 215, and thus may be weakly transferred to the tilting part 220, the height adjustment part 230, the movement part 240, or the body part 110. In this way, since the vibrations are weakly transferred to components other than the massage ball 212, durability of the massage device 100 and the massage module 200 may be improved.

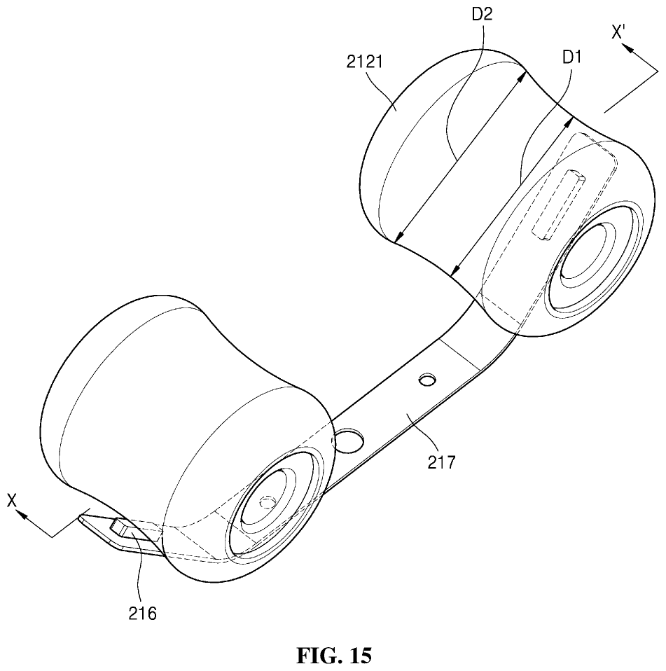

FIG. 15 is a view for describing an embodiment in which a temperature sensor 216 is disposed adjacent to the massage part 210 FIG. 8, and FIG. 16 is a cross-sectional view along line X-X′ of FIG. 15.

Referring to FIGS. 15 and 16, the massage part 210 according to one embodiment may further include a temperature sensor 216.

The temperature sensor 216 may be a part that measures the temperature of the massage ball 212. Thus, the temperature sensor 216 may measure the temperature of the massage ball 212, thereby adjusting a heating level provided to the body of the user.

As a selective embodiment, the temperature sensor 216 may measure the temperature of the massage ball 212 while in contact with the massage ball 212. Thus, the temperature sensor 216 may more precisely measure the temperature of the massage ball 212.

The massage part 210 according to one embodiment may further include a plate member 217 so that the temperature sensor 216 is stably in contact with the massage ball 212.

At least one region of the plate member 217 may be bent. For example, the plate member 217 may include a flat region, and both ends of the flat region may be bent. In this case, the bent region of the plate member 217 may be bent in a direction toward the massage ball 212.

The plate member 217 may be formed of an elastic material. Thus, the plate member 217 may be maintained bent in the direction toward the massage ball 212 by elasticity, and particularly, may be maintained in a direction surrounding the massage ball 212.

In this case, the temperature sensor 216 may be disposed at at least one end of the plate member 217, and the temperature sensor 160 may be maintained in contact with the massage ball 212 due to the elasticity of the plate member 217.

As a selective embodiment, the massage ball 212 may be formed such that a diameter D1 of a central portion thereof is smaller than a diameter D2 of the outside thereof. For example, the central portion of the massage ball 212 may be formed to be concave inward.

Thus, when the temperature sensor 216 and the plate member 217 are arranged adjacent to the massage ball 212, the volumes occupied by the temperature sensor 216, the plate member 217, and the massage ball 212 may be reduced. Further, since the temperature sensor 216 is disposed at a concave portion of the massage ball 212, the temperature sensor 216 may be prevented from being separated from the massage ball 212 while the massage ball 212 rotates.

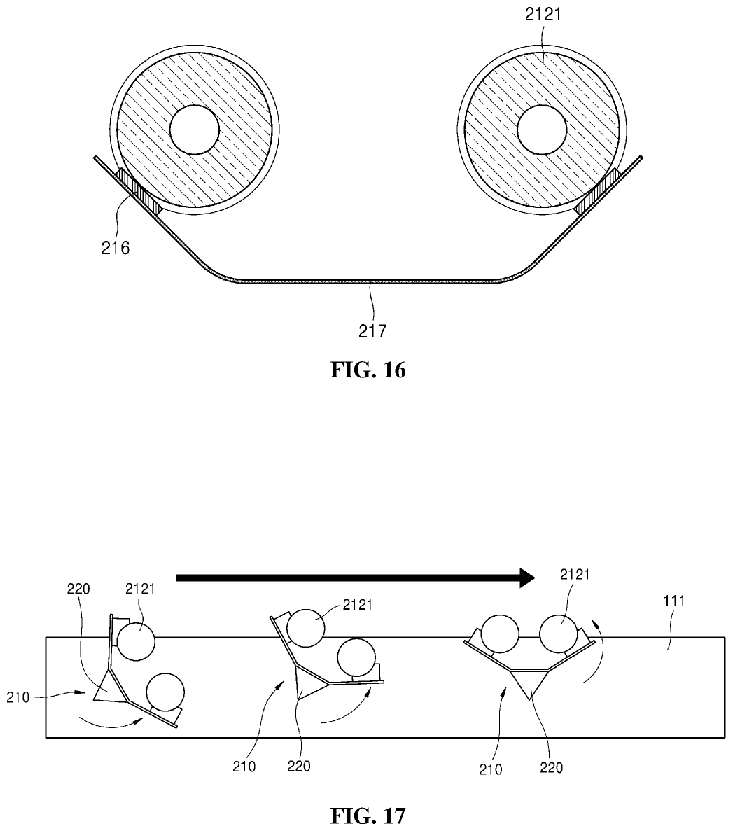

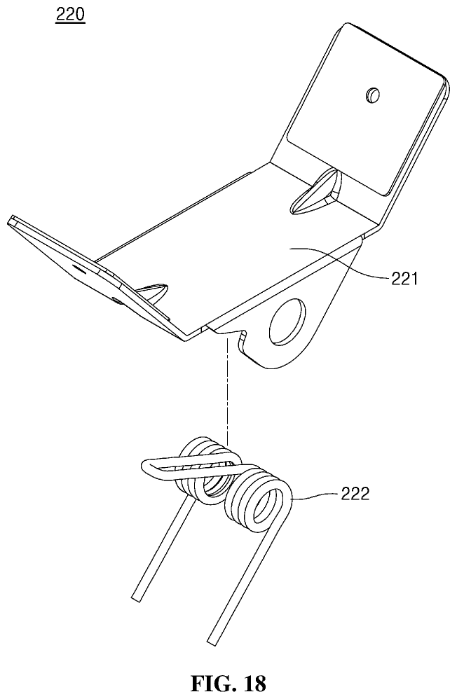

FIG. 17 is a view for describing one driving example of a tilting part 220 of FIG. 7, and FIG. 18 is an exploded perspective view of the tilting part 220 of FIG. 7.

Referring to FIGS. 17 and 18, the massage module 200 according to one embodiment of the present disclosure may further include the tilting part 220.

The tilting part 220 may be disposed on one side of the massage part 210 and control rotation of the massage part 210.

For example, the tilting part 220 may be coupled to the massage part 210. The tilting part 220 may control the rotation of the massage part 210 so that the massage part 210 is tilted to an appropriate angle. In detail, when a force is applied to the massage part 210 from the outside, the tilting part 220 may allow the massage part 210 to be tilted to an appropriate angle.

The tilting part 220 may be disposed on one side of the massage part 210 and may be disposed below the massage part 210 with respect to FIG. 7. Further, the tilting part 220 may be coupled to a lower side of the massage part 210 in the longitudinal direction. Here, the longitudinal direction may be a direction in which the massage module 200 moves along a massage groove.

The tilting part 220 does not automatically rotate the massage part 210 and may be tilted when an external force is applied to the massage part 210. For example, the tilting part 220 may control the rotation of the massage part 210 and may perform a buffering action against a force applied to rotate the massage part 210.

In detail, when a force is applied in a direction in which the massage part 210 rotates, and when the massage part 210 does not rotate to an extent in correspondence therewith, the massage part 210 may be damaged. Further, when the massage part 210 is caught by the body of the user while moving along the massage groove, and when the massage part 210 moves along a path without rotating at all, the massage part 210 strongly presses the body of the user, and the user may actually be injured.

In order to solve this problem, when a certain amount of force is applied to the massage part 210, the tilting part 220 may buffer the force so that the massage part 210 rotates to correspond to a direction of the force and may control the massage part 210 so as not to rotate beyond a predetermined angle.

As one embodiment, the tilting part 220 may include a tilting frame 221 and an elastic member 222.

The tilting frame 221 may be a component disposed below the massage part 210 and connected to the massage part 210. For example, the tilting frame 221 may be connected to the buffer part 215 below the massage part 210.

As a specific embodiment, the tilting frame 221 may be coupled to the lower side of the massage part 210 in the longitudinal direction, and this direction may be a direction in which the massage module 200 moves along the massage groove 111.

The tilting frame 221 may be coupled to a height adjustment bracket 231 which will be described below. For example, the tilting frame 221 may rotate about a separate shaft provided in the height adjustment bracket 231. To this end, the tilting frame 221 may have a through-hole into which the shaft provided in the height adjustment bracket 231 is to be inserted.

The elastic member 222 may be a part that is connected to the tilting frame 221 and provides elasticity for the rotational direction of the tilting frame 221.

When a rotational force is applied to the massage part 210, the elastic member 222 may be contracted to buffer the force. While the elastic member 222 is contracted, the tilting frame 221 may be tilted by a predetermined angle. Thereafter, when the force applied to the massage part 210 is weakened or eliminated, the elastic member 222 may return to its original location by the elasticity, and accordingly, the tilting frame 221 may return to its original location.

In this case, the elastic member 222 may be contracted and extended with respect the rotary axis about which the tilting frame 221 rotates. For example, the center of the contraction and expansion of the elastic member 222 and the rotary axis of the tilting frame 221 may be shared.

To this end, as illustrated in FIG. 16, the elastic member 222 may be provided with a through-hole into which the separate shaft provided in the height adjustment bracket 231 is to be inserted.

As a selective embodiment, the elastic member 222 may be a twisting spring or a torsion spring. When a general linear spring is used, a large space may be occupied inside the massage module 200. For example, in order to provide a sufficient angle and sufficient elasticity to the tilting frame 221, the linear spring should be disposed inside the massage module 200 by a predetermined length or more, but it is difficult for the massage module 200 to have a certain volume or more due to characteristics thereof. Thus, the elastic member 222, which is a twisting spring or a torsion spring, may directly provide elasticity in the rotational direction of the tilting frame 221, and thus may provide sufficient elasticity to the tilting frame 221 while occupying a minimum volume.

Referring back to FIG. 17, in a detailed description of one driving example of the tilting part 220, the tilting part 220 may be inclined at both ends of the massage groove 111. Thereafter, while the massage module 200 moves along the massage groove 111, the tilting part 220 may be gradually erected and then move in a horizontal state and may be tilted upon arriving at an opposite end in turn.

That is, the massage part 210 and the tilting part 220 may be inclined at a left end of FIG. 17, and the massage part 210 and the tilting part 220 may be erected at a right side of FIG. 17 while the massage module 200 moves.

As these components are included, the massage module 200 may provide a massage to correspond to the body of the user. That is, when the massage part 210 and the tilting part 220 are maintained in a horizontal state even when located at both ends of the massage groove 111, the user may feel discomfort because of a portion exposed through the massage groove 111 when lying on the body part 110. Thus, first, in a state in which the massage part 210 and the tilting part 220 are inclined, the user may comfortably lie on the body part 110, and thereafter, when a massage starts, the massage part 210 and the tilting part 220 may move while being gradually erected, to provide a massage to correspond to the body of the user.

Furthermore, the massage part 210 and the tilting part 220 may be tilted by a predetermined angle while moving in the direction of the height of the user. Since many parts of a human body are formed to be curved, the massage part 210 and the tilting part 220 may be tilted by a predetermined angle when reaching the curved parts of the body of the user while moving and may then be erected in the horizontal state after passing through the curved parts.

That is, the massage module 200 includes the tilting part 220, and thus may be flexibly tilted by a predetermined angle due to a force applied from the outside or an obstacle that interferes with progress, thereby smoothly and safely providing a massage.

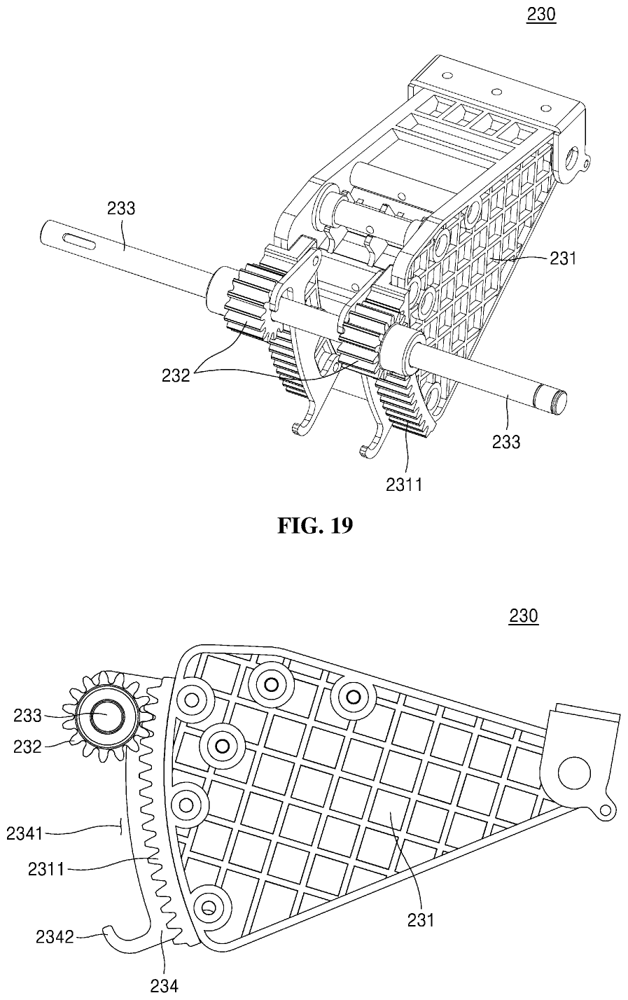

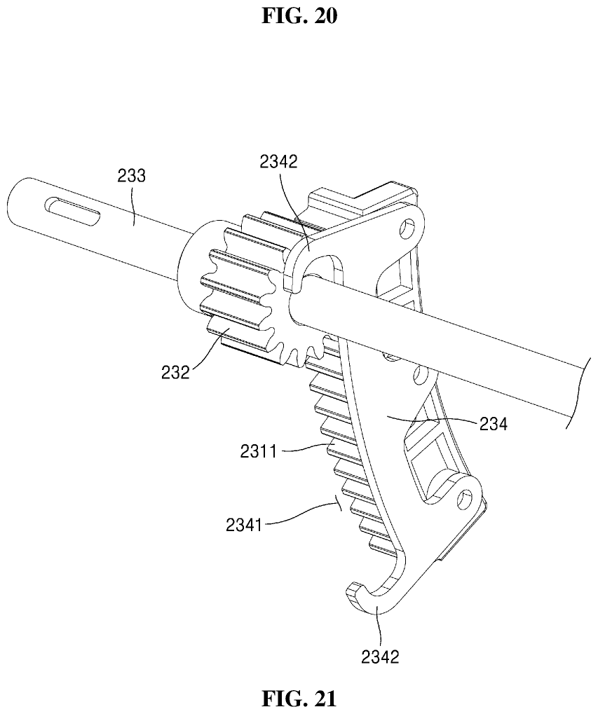

FIG. 19 is a view illustrating a height adjustment part 230 of FIG. 7, FIG. 20 is a view illustrating the height adjustment part 230 of FIG. 19 from a lateral side, and FIG. 21 is a view for describing a driving principle of the height adjustment part 230 of FIG. 19.

Referring to FIGS. 19 to 21, the height adjustment part 230 may be a part for adjusting the height of the massage part 210.

For example, the height adjustment part 230 may be connected to the tilting part 220. Further, the tilting part 220 may be connected to the massage part 210. Thus, the height adjustment part 230 may adjust the height of the connected tilting part 220, and at the same time, adjust the height of the massage part 210.

Accordingly, the height of the massage part 210 may be adjusted to correspond to the body of the user while the massage part 210 provides a massage to the user. Further, the massage part 210 may adjust the intensity of a massage applied to the body of the user while providing the massage to the user. For example, a force of acupressure on the body of the user, an intensity of applied vibration, or an intensity of tapping may be adjusted.

In detail, the height adjustment part 230 may adjust the heights of the massage part 210 and the tilting part 220 to correspond to the body of the user. For example, when a person lies on the upper surface of the body part 110, the waist is spaced apart from the upper surface as compared to the shoulders or the hips. Thus, to correspond to this feature, the height adjustment part 230 may lift up the massage part 210 when the massage part 210 passes through a location corresponding to the waist of the user while moving and may lower the massage part 210 when the massage part 210 passes through locations adjacent to the shoulders or locations adjacent to the hips.

Further, the height adjustment part 230 may adjust the heights of the massage part 210 and the tilting part 220 to correspond to the intensity of a provided massage. For example, the height adjustment part 230 may adjust the height of the massage part 210 to a high level to increase the intensities of acupressure, tapping, and vibration massages applied to the body of the user. In contrast, the height adjustment part 230 may adjust the height of the massage part 210 to a low level to decrease the intensities of acupressure, tapping, and vibration massages applied to the body of the user.

The height adjustment part 230 may include a height adjustment bracket 231, a first pinion gear 232, and a shaft member 233.

The height adjustment bracket 231 may form the overall shape of the height adjustment part 230. The first pinion gear 232 may be configured to vertically move the height adjustment bracket 231 while rotating, and the shaft member 233 may serve as a rotary shaft for a rotational movement of the first pinion gear 232.

As one embodiment, the height adjustment bracket 231 may be formed in a substantially fan shape. Further, the height adjustment bracket 231 may include a first rack gear 2311. For example, the height adjustment bracket 231 may include a first rack gear 2311 formed in a circumferential direction of the fan shape.

In this case, the first rack gear 2311 may be formed as a separate member and then be coupled to the height adjustment bracket 231 or may be formed integrally with a circumference of the height adjustment bracket 231.

The first pinion gear 232 may rotate while engaged with the first rack gear 2311. For example, as the first pinion gear 232 rotates, the first rack gear 2311 engaged with the first pinion gear 232 may move, and accordingly, the height adjustment bracket 231 may rotate together.

In detail, when the first pinion gear 232 rotates about the shaft member 233, the first rack gear 2311 engaged with the first pinion gear 232 may move to correspond to a rotational direction of the first pinion gear 232.

In description with reference to FIG. 20, when the first pinion gear 232 rotates in a counterclockwise direction, the first rack gear 2311 may move upward to correspond to the rotation of the first pinion gear 232, and accordingly, the height adjustment bracket 231 may be lifted upward.

In contrast, when the first pinion gear 232 rotates in a clockwise direction, the first rack gear 2311 may move downward to correspond to the rotation of the first pinion gear 232, and accordingly, the height adjustment bracket 231 may be lowered downward.

As a result, the heights of the massage part 210 and the tilting part 220 may be adjusted.

As one embodiment, the height adjustment part 230 may include a rotation actuator that provides rotational power to the first pinion gear 232.

The rotation actuator may rotate the first pinion gear 232. In this case, the amount and intensity with which the rotation actuator rotates the first pinion gear 232 may be controlled by the controller. Further, an input signal for controlling the first pinion gear 232 may be input by the user.

As a selective embodiment, the height adjustment bracket 231 may further include a guider 234 extending in a lengthwise direction of the first rack gear 2311.

The guider 234 may guide a relative movement path of the height adjustment bracket 231 and the shaft member 233. In a detailed description with reference to FIG. 21, the guider 234 may be formed in parallel with the first rack gear 2311.

A guide groove 2341 serving as a path through which the shaft member 233 moves may be formed inside the guider 234. However, in detail, since the height adjustment bracket 231 provided with the first rack gear 2311 is moved by rotating the first pinion gear 232, the guide groove 2341 may guide the relative movement path of the shaft member 233 and the height adjustment bracket 231.

As a selective embodiment, a stopper 2342 may be formed at at least one end of the guider 234. As a preferable embodiment, the stoppers 2342 may be formed at both ends of the guider 234.

The stopper 2342 may prevent the shaft member 233 from being separated from the guide groove 2341. For example, when the first rack gear 2311 moves beyond a limit, the first rack gear 2311 may be separated from the first pinion gear 232. Thus, when the first rack gear 2311 moves to a limit point, the shaft member 233 may be stopped by the stopper 2342, and the height adjustment bracket 231 may be prevented from moving beyond the limit point.

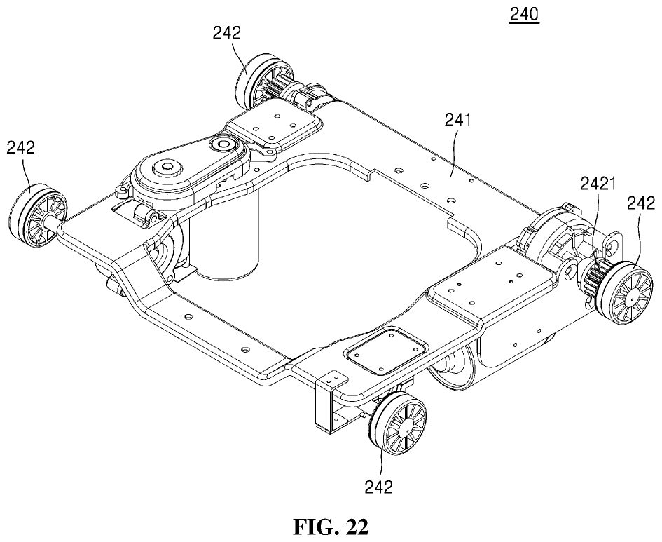

FIG. 22 is a view illustrating a movement part 240 of FIG. 7.

The movement part 240 may be a part that moves the massage module 200.

Referring to FIG. 22, the movement part 240 may include a movement frame 241 and a movement wheel 242.

The movement frame 241 may form an overall frame of the movement part 240.

A space for accommodating the height adjustment part 230 may be provided inside the movement frame 241. The height adjustment part 230 may be accommodated in the space provided in the height adjustment part 230. In this case, the height adjustment part 230 may be connected to the tilting part 220, and the tilting part 220 may be connected to the massage part 210. Thus, the movement part 240 may move the accommodated height adjustment part 230 to move the tilting part 220 and the massage part 210 connected to the height adjustment part 230 at the same time. As a result, the movement part 240 may allow the entirety of the massage module 200 to move.

In detail, the massage module 200 may be moved linearly inside the massage device 100 by the movement part 240. For example, the massage module 200 may move inside the body part 110 along the massage groove 111 in a lengthwise direction of the height of the user. Thus, the massage module 200 may provide a massage to various parts of the body of the user while moving in the lengthwise direction of the body of the user.

The movement wheel 242 may be installed on the movement frame 241 to allow the movement part 240 to move inside the body part 110. For example, the movement wheel 242 may be provided as a plurality of movement wheels 242 and may be arranged on both side surfaces of the movement frame 241. The movement wheel 242 may roll inside the body part 110 to move the massage module 200.

As a selective embodiment, the movement part 240 may further include a second pinion gear 2421 located adjacent to the movement wheel 242. For example, the second pinion gear 2421 may share a rotary shaft with any one of the movement wheels 242. That is, the second pinion gear 2421 may simultaneously rotate together with any one of the movement wheels 242.

Although not illustrated, a second rack gear engaged with the second pinion gear 2421 may be formed inside the body part 110. For example, the second rack gear may be formed inside the body part 110 along the massage groove 111.

Accordingly, the second pinion gear 2421 may move along the second rack gear while rotating, and accordingly, the movement wheel 242 may rotate together, so that the movement part 240 may move along the massage groove 111.

As one embodiment, the massage module 200 may further include a part that provides rotational power to the second pinion gear 2421. For example, the part that provides the rotational power to the second pinion gear 2421 may be a motor or an actuator.

Thus, the second pinion gear 2421 may rotate by receiving the rotational power, and accordingly, the movement part 240 may linearly move along the massage groove 111.

The present disclosure can provide a massage module that, when a force is applied to the massage module, can provide a safe massage to the body of a user by buffering the force.

The present disclosure can provide a massage module of which the height and angle can be adjusted to correspond to the body of the user.

However, the effects of the present disclosure are not limited to the above-described effects and include effects that can be easily understood by those skilled in the art to which the present disclosure pertains from the present specification and the accompanying drawings.

Although embodiments of the present disclosure have been described above in more detail with reference to the accompanying drawings, the present disclosure is not necessarily limited to these embodiments, and various modifications may be implemented without departing from the technical spirit of the present disclosure. Thus, the embodiments disclosed in the present disclosure are not intended to limit the technical spirit of the present disclosure, but are intended to describe the present disclosure, and the scope of the technical spirit of the present disclosure is not limited by these embodiments. Therefore, it should be understood that the embodiments described above are not limiting but illustrative in all aspects. The scope of protection of the present disclosure should be interpreted with reference to the appended claims, and all technical spirits within the scope equivalent thereto should be interpreted as being included in the scope of the present disclosure.

What is claimed is:

1. A massage module having an improved degree of freedom, comprising:
a massage part that provides a massage to a body of a user;
a tilting part that is disposed on one side of the massage part and controls rotation of the massage part; and
a height adjustment part that is connected to the tilting part and adjusts a height of the massage part,
wherein the massage part includes at least one buffer part disposed at a portion of the massage part that is directly coupled to the tilting part.

2. The massage module of claim 1, wherein the tilting part includes:
a tilting frame connected to the buffer part; and
an elastic member that controls rotation of the tilting frame.

3. The massage module of claim 2, wherein the elastic member is a torsion spring that is pressed while rotating about a rotary axis of the tilting frame.

4. The massage module of claim 1, wherein the height adjustment part includes:
a height adjustment bracket;
a first rack gear provided on the height adjustment bracket;
a first pinion gear that rotates while engaged with the first rack gear; and
a rotation actuator that provides rotational power to the first pinion gear.

5. The massage module of claim 4, wherein the height adjustment bracket includes a guider extending in a lengthwise direction of the first rack gear.

6. The massage module of claim 5, wherein the guider has a stopper formed at one end thereof.

7. The massage module of claim 1, wherein the buffer part includes a damper member of which a shape is deformed by a force applied to the massage part.

8. The massage module of claim 7, wherein the buffer part includes a damper shaft that is disposed to pass through the damper member and has one end extending inward from the massage part and the other end extending to the tilting part.

9. The massage module of claim 1, further comprising a movement part that moves the massage part, the tilting part, and the height adjustment part accommodated in a space that is provided for accommodating the height adjustment part.
